(19) United States
(12) Patent Application Publication
(10) Pub. No.: US 2006/0057084 A1
(43) Pub. Date: Mar. 16, 2006

(54) Attero-chromic color aggregates

(76) Inventor: Anthony D. Gonzalez, Oak Ridge, NJ (US)

Correspondence Address: Charles J. Zeller, Esq.;Avon Products Inc., Avon Place, Suffern, NY, 10901, US

(21) Appl. No.: 10/940,450

(22) Filed: Sep. 14, 2004

(51) Int. Cl. A61K 8/81; A61K 8/19

(52) U.S. Cl. 424/63

(57) ABSTRACT

Compositions are provided which change color or modify a color characteristic of a personal care product in response to the application of mechanical energy, such as is provided by the user during application of the product. Methods of making color-changing compositions and personal care products incorporating color-changing compounds are also disclosed.

### FIELD OF INVENTION

The present invention relates generally to irreversible color-changing compositions useful in personal care products. More particularly, the present invention relates to compositions which change color in response to the application of mechanical energy to the composition.

### BACKGROUND OF THE INVENTION

There has been increasing interest in formulating a variety of cosmetic products that change color in response to stimuli such as light, heat, pH change, rubbing, etc. Such color-changing products provide a visually compelling experience to the user and further the range of effects achievable with color cosmetics.

In addition to color cosmetics, color-changing compositions have found utility in a variety of personal care, consumer and medicinal products. One interesting application of color-changing compositions is as a calorimetric indicator indicative of, for example, the extent of application of a product or the continued presence of a product on the skin. For example, some sun-care products use color as an indicator to determine whether the product has been initially applied over the desired skin areas. Some such products go on colored and become invisible as they are rubbed into the skin. For example, U.S. Pat. No. 6,086,858 (McEleney et at.), U.S. Pat. No. 6,007,797 (Bell et al.), U.S. Pat. No. 6,146,618 (Bell et al.), U.S. Pat. No. 6,099,825 (McShane et al.), U.S. Pat. No. 6,290,936 B1 (Ross et al.), and U.S. Pat. No. 5,747,011 (Ross et al.) disclose sun-care products which exhibit a visible color when applied to the skin as an indication of the extent of coverage. The color subsequently fades or becomes clear by virtue of a pH change, such as brought about by contact with the skin.

Compositions are known in the art which take advantage of a variety of phenomena to create color changes. For example, U.S. Pat. No. 6,733,766 to Gott discloses compositions comprising water soluble dyes formulated with an oil soluble carrier in which the pigments are insoluble. The dry composition does not exhibit the color of the pigment but upon application of water to the composition, as for example during skin cleansing, color is developed.

As discussed above, another approach in the art for providing color changing compositions has been to employ pigments or dyes which undergo a color change in response to a change in pH. For example, U.S. Pat. No. 5,523,075, U.S. Pat. No. 5,532,029, U.S. Pat. No. 5,837,645, U.S. Pat. No. 5,997,891, and U.S. Pat. No. 6,139,821, each to Fuerst et al., disclose compositions, such as sun-care cosmetic compositions, that exhibit color changes in response to a pH change. The pH change is mediated by an ingredient which alters pH following application to the skin and typically evaporates or degrades thereafter.

Other compositions and methods known in the art employ materials which change color in response to temperature changes (i.e., “thermochromic” materials) and/or light (i.e., “photochromic” materials).

U.S. Pat. No. 6,290,977 to Friars et al. provides flowable personal care compositions having a thermochromic pigment, and a methodology for sensing or recording the temperature of skin using compositions having a thermochromic pigment.

Inorganic photochromic compounds, such as silver halides, are well known in the art. For example, U.S. Pat. No. 6,306,409 to Ogawa et al. discloses photochromic cosmetic compositions comprising titanium oxide coated mica. The use of inorganic photochromic pigments has traditionally been limited, however, as organic compounds generally show more sensitive color changes between colored and colorless states and are generally more suitable for addition to organic compositions than are conventional inorganic photochromic compounds. More recently, a wide variety of organic photochromic compositions have been developed. For example, U.S. Pat. No. 6,531,118 to Gonzalez et al. provides topical compositions having reversible visible color change in response to changes in light intensity. The disclosed photochromic materials are useful in a variety of personal care products, for instance, as an indicator of protection against ultraviolet radiation in a sunscreen product.

The compositions and methods described above generally require an external stimulus, such as light or heat, or the presence of a pH-modifying agent to affect a change color. In some applications (i.e., indoors or under poor lighting conditions, application to cool surfaces, etc.), the required stimulus may not always be available. Further, the user has little or no control over the timing or extent of color development or change.

Another approach to providing color changing materials has been to employ materials that develop or change color in response to a mechanical stimulus, such as rubbing. The applicant has termed this phenomenon the “attero-chromic effect” (from the Latin “attero” meaning “to rub”). Heretofore, this approach has generally been limited in the cosmetic field to the use of frangible pigment-containing microcapsules. U.S. Pat. No. 4,756,906 to Sweeny discloses cosmetic compositions containing a first pigment and microcapsules containing a liquid and a second pigment. Upon rupture of the microcapsules, such as by rubbing, the coloration of the second pigment is added to the composition, altering the color characteristics provided by the first pigment. WO 01/35933 similarly discloses materials comprising miscroencapsulated pigments for cosmetic and dermal compositions such as make-up. In one embodiment, a double-layered microcapsule is provided having an inner core containing iron oxides and an outer layer containing titanium dioxide. Upon application and subsequent rupture of the white microcapsules, a brown color is released. The applicability of this approach is somewhat limited as microcapsules are sensitive to aqueous processing conditions that traditional emulsion cosmetic products undergo during manufacture.

Similar to the microcapsule-type color indicators discussed above are the color-indicating beads disclosed in U.S. Pat. No. 6,309,655 to Minnix. Minnix discloses a self-heating facial mask comprising “self-indicating granules” which are readily friable, such that the physical manipulation of applying the cosmetic composition to wet skin is sufficient to disintegrate the granules and release the colorant. The self-indicating granules comprise a water-insoluble polymer and a water-dispersible colorant. The color indicator may take the form of an “agglomeration” of water-insoluble polymer particles held together by a binder and containing a colorant. Alternatively, the color indicator material may take the form of a water-insoluble polymer matrix in which the colorant is encapsulated or entrapped. The preferred water-insoluble granules are made of polyethylene or polymethacrylate copolymer. The disclosed colorants suitable for use include inorganic pigments such as ultramarine, TiO₂, iron oxide and chromium green. The product is provided as an anhydrous cosmetic composition. Due to their similarity to microcapsule-type compositions, it is believed that the beads disclosed in Minnix will exhibit similar sensitivity to aqueous processing conditions that traditional emulsion cosmetic products undergo during manufacture and storage, especially in the presence of both oil and water components.

In view of the desirability of personal care products formulated to provide a color change in response to a stimulus, there is a continuing need to expand the range of color-changing compositions in order to provide compositions suitable for use in a larger array of products and applications. In this regard, there is a need for products that exhibit color-change in response to the application of mechanical energy, such as by rubbing or application of the product with the fingers or an applicator. Further, there is a need for compositions that exhibit color-changing phenomena that are stable under the processing and storage conditions to which cosmetics are subjected.

It is therefore an object of the present invention to provide compositions suitable for use in personal care products, such as cosmetics, that produce an irreversible visible color change affected by the application of mechanical energy (i.e., an attero-chromic effect) such as provided by the fingers or palms, by means of an applicator, or otherwise, during application of the product.

It is a further object of the invention to provide compositions that produce a color change upon application of mechanical energy which are readily formulated into and stable in a variety of personal care products, such as those employing oil-in-water and oil-in-water emulsions.

### SUMMARY OF THE INVENTION

In accordance with the foregoing objectives, the present invention provides color-changing compositions and products as well as methods for making color-changing compositions and products.

One aspect of the invention provides color-changing compositions comprising a color aggregate. The color aggregate according to this aspect of the invention comprises an anionic polymer and an optionally surface treated non-encapsulated metal oxide colorant non-covalently associated with the polymer. The anionic polymer is at least partially neutralized with a neutralizing agent.

The anionic polymers are water dispersible at a first pH and water soluble at a second higher pH. In one embodiment, the anionic polymers are preferably water-dispersible at a pH of less than about 5 and water soluble at a pH between about 5 and about 7.

In another embodiment of the invention, it is preferred that the metal oxide colorants are selected from those having a positive zeta potential in acidic media. It is further desirable that the metal oxide colorant has a positive zeta potential and the anionic polymer has a negative zeta potential at a particular pH between about 3 and about 7, although not necessarily over this entire range.

It is also preferred in all embodiments of the invention that the color changing composition comprise an aggregate of an at least partially neutralized anionic polymer associated with a metal oxide colorant through ionic interactions.

One currently preferred color-changing composition according to this aspect of the invention comprises an anionic acrylates/C10-30 alkyl acrylate crosspolymer, a non-encapsulated optionally surface treated metal oxide colorant selected from the group consisting of titanium dioxide, iron oxides, and combinations thereof. The anionic polymer is at least partially neutralized with a neutralizing agent such as an amine.

Another aspect of the invention provides personal care products, such as, for example, color cosmetics and sun-care products, which incorporate the inventive color-changing compositions. Since the color-changing compositions of the present invention are substantially stable under aqueous processing and storage conditions, they are ideally suited for incorporation in personal care products comprising aqueous emulsions.

Yet another aspect of the invention provides a method for making color-changing compositions. Generally, the method comprises the steps of: (1) providing an aqueous slurry having a first acidic pH, preferably less than about 5, the aqueous slurry comprising an anionic polymer and a metal oxide colorant; and (2) adding a neutralizing agent to the aqueous slurry in an amount sufficient to raise the pH of the aqueous slurry to a second pH, preferably between about 5 and 7.

The anionic polymer is preferably water-dispersible at a pH of less than about 5 and water soluble at a pH between about 5 and about 7. The anionic polymer typically comprises between about 0.1 to about 5% by weight of the aqueous slurry.

The metal oxide colorants are preferably selected from those having a positive zeta potential in acidic media. It is further desirable that the metal oxide colorant has a positive zeta potential and the anionic polymer has a negative zeta potential at a particular pH between about 3 and about 7, although not necessarily over this entire range.

A currently preferred method of making a color changing composition according this aspect of the invention comprises the steps of: (1) providing an aqueous slurry having a first pH, preferably less than about 5, wherein the aqueous slurry comprises (a) an anionic acrylates/C10-30 alkyl acrylate crosspolymer which is water-dispersible at the first pH (i.e. preferably less than about 5) and water soluble at a second pH, preferably between about 5 and about 7, (b) an optionally surface treated metal oxide colorant selected from the group consisting of titanium dioxide, iron oxides, and combinations thereof, and (c) an electrolyte; and (2) adding a neutralizing agent comprising an amine to the aqueous slurry in an amount sufficient to raise the pH of the aqueous slurry from the first pH to the second pH. The anionic polymer typically comprises between about 0.1 to about 5% by weight of the aqueous slurry and the electrolyte typically comprises between about 0.1 to about 3% by weight of the aqueous slurry.

These and other aspects of the invention will be better understood by reference to the following detailed description and appended claims.

### DETAILED DESCRIPTION OF THE INVENTION

Generally, the present invention provides color-changing compositions which are initially substantially colorless, weakly colored, or have an initial first color before or immediately after application to a surface such as skin or hair, and subsequently develop a color, a more intense color, or a second color upon the application of mechanical energy, such as is provided by the user during application of the composition.

While not generally recognized in the art, the applicant has coined the term “attero-chromic effect” to describe this phenomenon. As used herein, the term attero-chromic embraces any phenomenon wherein a composition displays a change in any color characteristic upon the application of mechanical energy to the composition. The change in color characteristic may be, for example, a change in hue, intensity, lightness or darkness, saturation, interference, etc. The effect obtained by the compositions of the present invention cannot be reversed, and are unaffected by light or heat. Typically, the attero-chromic effect is brought about by mechanical energy supplied externally, such as, for example, by a persons hands, palms, fingers, or an applicator, etc. The term is not so limited, however, and embraces any source of mechanical energy. Generally, the attero-chromic color-changing compositions of the invention comprise an anionic polymer and a colorant, such as a pigment or lake.

Without wishing to be bound by any theory, it is believed that the colorant is associated with the anionic polymer through non-covalent interactions such as ionic bonds. Without wishing to be bound to any particular §tructure or mechanism of interaction, the polymer-colorant association is referred to for convenience herein as an “aggregate.” The structure of the aggregate is such that it prevents the pigment from dispersing in the vehicle such that the color of the pigment is not fully observed. However, the colorant is not chemically altered by association with the polymer. When mechanical energy is applied to the aggregate in comparable or greater magnitude than the energy of the non-covalent interactions which associate the colorant with the anionic polymer, the colorants dissociate from the anionic polymer producing a visible color change to the surround medium.

The inorganic pigments of the invention are below their isoelectric point at low pH. That is, at acidic pH the pigment particles have a positive zeta potential and thus the surface of the pigment particle carriers a net positive charge. Preferably, the zeta potential of the inorganic pigment is positive at pH of less than about 5. For example, the zeta potential of commercial grades of rutile titanium dioxide typically varies from about 50 mv at a pH of about 3.5 to about −30 mv at a pH of about 11. The isoelectric point of titanium dioxide is typically between 6 and 8. Thus, at low pH (i.e. below the isoelectric point) it is believed that the metal oxide colorants are subject to a an attraction such as, for example, a coulombic attraction, to the anionic polymer which carrier a net negative charge even at low pH. Upon neutralization, it is believed that the interaction between the polymer and the metal oxide colorant persists. That is, the metal oxide pigments are not displaced from the surface of the anionic polymer by salt formation.

It is surprising, and heretofore unknown, that an aggregate of metal oxide pigment and anionic polymer formed at low pH persists at conditions approaching neutral or even basic pH and is capable of producing an attero-chromic effect. In fact, it is typical in the art of cosmetic formulation to neutralize anionic polymers before addition of metal oxide pigments. In such prior compositions, attero-chromic phenomena are not observed, suggesting that aggregates are not formed.

1. Attero-Chromic Compositions

One embodiment of the invention is a color-changing composition comprising a water-soluble anionic polymer, a non-encapsulated colorant, and a neutralizing agent. Suitable materials for each of these components are provided below.

Water-Soluble Anionic Polymers

Examples of suitable water-soluble anionic polymers include, but are not limited to carbomers, polyacrylates, acrylates copolymers, organo-modified acrylates copolymers containing from 3 to 60 carbons, vinyl acrylates, acrylamide polymers and copolymers, and combinations thereof.

Currently preferred water-soluble anionic polymers include Carbopol® ETD 1342 (Noveon, Ohio) and Carbopol® ETD 2020 (Noveon, Ohio) which are acrylates/C10-30 alkyl acrylate crosspolymers according International Nomenclature for Cosmetic Ingredients (INCI).

The anionic polymers of the invention are preferably water-dispersible at a pH of less than about 5 and water soluble at a pH of between about 5 and about 7 may by used in the practice of the invention. While it is desirable for the practice of the invention that the anionic polymers are soluble at a pH of between about 5 and about 7, the preferred polymers may be water soluble at pH above about 7 as well. The terms “water dispersible” and “water soluble” are used according to their well known meanings in the art.

Preferred anionic polymers have a negative zeta potential at an acidic pH, preferably below about 6 and more preferably below about 5.

Colorants

Colorants of the present invention are metal oxide pigments and lakes. While the specific embodiments discussed herein employ inorganic pigments, it is contemplated that some organic pigments and lakes will be useful in the practice of the invention. The invention embraces the use of both individual pigments and combinations of pigments. Thus, the term “colorant” includes individual pigments and combinations of pigments. It is within the skill in the art to choose pigments and combinations of pigments to produce a desired color or change in a color characteristic. Further, the colorant may be a composition which does not alone impart color but rather alters a color characteristic of the composition, such as pearling agents and the like.

Exemplary inorganic pigments are metal oxides and metal hydroxides such as magnesium oxide, magnesium hydroxide, calcium oxide, calcium hydroxides, aluminum oxide, aluminum hydroxide, iron oxides (α-Fe₂O₃, y-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides and composite oxides and composite hydroxides such as iron titanate, cobalt titanate and cobalt aluminate. Other suitable colorants include, Prussian blue, iron blue, iron hexacyano ferrate, bismuth oxychloride, titanated mica, iron oxide titanated mica, and the like. Other colorants are disclosed in U.S. Pat. No. 5,340,569 (Elliot et al.) and U.S. Pat. No. 6,692,754 (Makimoto et al.), the contents of which are hereby incorporated by reference. Examples of pearling pigments are bismuth oxychloride, guanine, and titanium composite materials containing, as a titanium component, titanium dioxide, titanium lower oxides or titanium oxynitride, as disclosed in U.S. Pat. No. 5,340,569 (Elliot et al.), the contents of which are hereby incorporated by reference.

The inorganic metal oxide pigments of the invention are preferably below their isoelectric point and have a positive zeta potential at an acidic pH, preferably the zeta potential of the inorganic pigment is positive at pH of about 5, about 4.5, about 4, about 3.5, and/or about 3.

The selection of pigments to suit the appropriate application is within the skill of the ordinary artisan. Of course, where the attero-chromic composition is employed in a personal care product, such as a cosmetic or sun-care product, only those pigments compatible with personal care products should be used.

The preferred pigments according to the invention are titanium dioxide and iron oxides.

By “non-encapsulated” colorant, it is meant that the colorant is not associated with the shell or lumen of a polymeric microcapsule or entrapped in a polymer matrix. Such encapsulated pigments are well known in the art and are disclosed, for example, in U.S. Pat. No. 4,756,906 and WO 01/35933, the contents of which are hereby incorporated by reference. The term “non-encapsulated” does not preclude the use of surface treated colorants in the practice of the invention; the distinction between encapsulated colorants and surface treated colorants being well understood in the art.

According to one embodiment of the invention, one or more inorganic pigments comprising the colorant is surface treated to modify the hydrophilic/hydrophobic character of the pigment. Suitable material for surface treatment include, but are not limited to, alumina, silica, fumed silica, waxes containing from 3 to 60 carbons, silicones, organosilicones, halosilicones, fluorocarbons, amino acids and salts and esters thereof, olefins and polymers thereof, fomblins, mineral oil, fatty acids and derivatives thereof, fatty alcohols and derivatives thereof, soaps, metal oxides, inorganic salts, halogenated organic compounds, surfactants, phospholipids, hydrogenated lecithin, collagen, carbonate esters and ethers and polymers and copolymers thereof. Preferred surface treatment materials include silicones, and particularly methicone (methylhydrogenpolysiloxane) and dimethicone. Surface treated inorganic pigments and methods for surface treating inorganic pigments are well known in the art and are disclosed in, for example, U.S. Pat. No. 5,486,631 (Mitchnick et al.), U.S. Pat. No. 5,368,639 (Hasegawa et al.), U.S. Pat. No. 4,622,074 (Miyoshi et al.), U.S. Pat. No. 5,326,392 (Miller et al.), and U.S. Pat. No. 4,606,914 (Miyoshi et al.), the contents of which are hereby incorporated by reference.

Surface treatment of inorganic pigments may offer advantages such as increased water-repellency, improved skin adhesion and wear properties in cosmetic products as well as improved handling and manufacturing characteristics. Further, surface treatment may alter the zeta potential of the pigment, allowing the skilled artisan to increase or decrease the isoelectric point and zeta potential at any desired pH.

The preferred surface treated pigments according to one embodiment of the invention are titantium dioxide and iron oxides treated with silicones, more preferably methicone.

Any amount of colorant may be employed. However, it is preferred to use only so much colorant as will form an aggregate with the anionic polymer to avoid having substantial amounts of colorant dispersed in the aqueous phase which may minimize the visual effect of the attero-chromic color change. In this regard, the weight ratio of colorant to anionic polymer is preferably from about 30:1 to about 1:1. More preferably, the weight ratio of colorant to anionic polymer is from about 15:1 to about 5:1. It is within the skill in the art to select a ratio of colorant to polymer to account for factors such as the molecular weight of the colorant and polymer and the desired intensity of the attero-chromic effect.

Neutralizing Agents

The anionic polymer/colorant aggregates according to the invention are neutralized prior to use. Neutralization is necessary to render the polymer water-soluble and to provide a product having a physiologically compatible pH. Consequently, the color changing compositions of the invention will comprise a neutralizing agent. Without wishing to be bound by any theory or limited to any particular structure, it is believed that the neutralizing agent is associated with the anionic polymer, typically in the form of a salt of the polymeric acid groups.

Suitable and preferred neutralizing agents are discussed in detail in the method section that follows.

According to the forgoing preferred embodiments, the most preferred color-changing compositions according to the invention comprise a color-changing composition comprising an anionic acrylates/C10-30 alkyl acrylate crosspolymer, a non-encapsulated optionally surface treated colorant selected from the group consisting of titanium dioxide, iron oxides, and combinations thereof, and a neutralizing agent comprising an amine.

2. Method of Preparing Attero-Chromic Compositions

The attero-chromic color-changing compositions of the invention are generally made by a process comprising the steps of: (1) providing an aqueous slurry having a pH of less than about 5 comprising an anionic polymer and a metal oxide colorant; and (2) adding a neutralizing agent to the aqueous slurry in an amount sufficient to raise the pH of the aqueous slurry to between about 5 and 7.

The anionic polymer is water-dispersible at a pH of less than about 5 and water soluble at a pH of between about 5 and about 7. Suitable and preferred water-soluble anionic polymers and colorants are those previously described herein.

The aqueous slurry is preferably prepared by adding the anionic polymer and one or more optionally surface treated pigments to water in a suitable vessel for mixing. The anionic polymer is preferably added to water first. The anionic polymer is preferably added in an amount sufficient to react with the desired amount of pigment. It is contemplated that acids such as, for example, hydrochloric acid, may be added to the water if the polymer alone is not present in sufficient quantity to lower the pH below about 5. The anionic polymer will preferably comprise between about 0.1 to about 5% by weight, and more preferably between about 0.25 and about 1.5% by weight of the aqueous slurry. This amount may vary within these ranges, depending on factors such as the hydrophilicity of the polymer, the molecular weight of the polymer, the surface area of the aggregate, and the presence of electrolytes. The skilled artisan may determine the appropriate amount of a specific anionic polymer through routine experimentation.

The invention imposes no restriction on the amount of pigment that may be added to the slurry.

The aqueous slurry is mixed at a high rate of agitation. Preferably, the mixing speed is selected to create a vortex or a deep vortex. After the pigments have become dispersed in the aqueous slurry, a neutralizing agent is added to the slurry. The neutralizing agent may be any agent capable of raising the pH of the slurry to above about 5. Suitable neutralizing agents include, but are not limited to, amines, amino acids, ammonium compounds, hydroxides of alkali and alkaline earth metals, and combinations thereof. Preferred neutralizing agents are amines. The most preferred neutralizing agents are mono-, di-, or tri-hydroyalkyl amines such as triethanolamine. The neutralizing agent is added to the slurry in an amount sufficient to raise the pH of the slurry from a first acidic pH, preferably below about 5, to a second pH, preferably above about 5, and more preferably about 7.

Upon addition of the neutralizing agent, mixing is continued until the composition is substantially uniform. The anionic polymer/color aggregates that form may be isolated by any method known in the art such as by evaporation of water or filtration. The isolated aggregates may be incorporated into a personal care product as described herein. Preferably, however, the aggregates are not isolated and the aqueous composition is directly incorporated into a product. Where the aggregates are not isolated, it may be desirable, but not necessary, to add electrolytes to the aqueous mixture in order to facilitate the incorporation of the color aggregates into emulsion-type products.

There is no limitation on the type of electrolyte that may optionally be used. Suitable electrolytes include, but are not limited to, salts of alkali or alkaline earth metals, and combinations thereof. Currently preferred electrolytes are NaCl, KCl, CaCl₂, MgCl₂, and combinations thereof.

The electrolytes may be added before or after neutralization. It has been found that the incorporation of electrolytes before neutralization may advantageously serve to increase the amount of anionic polymer that may be employed. When used, electrolytes preferably comprise less than about 3% by weight, and more preferably less than about 0.5% by weight of the aqueous slurry.

3. Products Comprising Attero-Chromic Compositions

The attero-chromic compositions of present invention are contemplated to be useful for any application where it is desired to produce a color change in response to the application of mechanical energy. Currently preferred uses for the attero-chromic compositions of the invention are in personal care products that provide a surface coating to skin, such as cosmetics, lotions, etc. These products include, but are not limited to color cosmetics, self-tanning lotions, skin treatments and skin care products.

One contemplated use is in a sun-care product such as sunscreens. A sunscreen may be formulated to be colorless, white, or having a first color before the product has been applied to the skin. As the user applies the sunscreen incorporating the inventive attero-chromic compositions to the skin, the mechanical energy imparts a tan look to the skin. It is within the skill in the art to select pigments that can produce a brownish hue resembling tanned skin. Accordingly, a user may be motivated to frequently apply the product in order to achieve the appearance of a tan. Further, the effect of the lotion changing from colorless or white to colored is a visually appealing phenomenon which may further motivate individuals, in particular children, to apply sunscreen before going outdoors.

Cosmetically Acceptable Vehicles

Many commercial cosmetic products typically take the form of an emulsion, which is the preferred cosmetic vehicle according to the invention.

An emulsion is a mixture of two immiscible liquids in which droplets of one liquid (the discontinuous or internal phase) are dispersed throughout the other liquid (the continuous or external phase). Emulsions may behave and appear as substantially homogenous fluids. The products of the present invention comprise a cosmetically acceptable vehicle in the form of an emulsion, including but not limited to, oil-in-water, water-in-oil, oil-in-oil, polyol-in-oil emulsions. An oil-in-water emulsion is a mixture where water-insoluble droplets are dispersed in a continuous aqueous phase. Conversely, a water-in-oil emulsion is a mixture where water droplets form the discontinuous phase which is dispersed in a continuous oily phase.

Other emulsions such as water-in-silicone, silicone-in-water, and silicone-in-oil emulsions are also contemplated to be useful in the practice of the invention.

Multiple phase emulsions such as water-in-oil-in-water and oil-in-water-in-oil emulsions are also contemplated to be useful according to the invention. These emulsions comprise droplets of an emulsion dispersed in another emulsion. For example, a water-in-oil-in-water emulsion comprises a continuous water phase containing dispersed oil droplets which is itself dispersed in a continuous oil phase. Double emulsions and multiple emulsions are also contemplated to be useful in the practice of the invention.

The preferred products according to the present invention comprise an oil-in-water emulsion as the cosmetically acceptable vehicle.

The aqueous phase preferably comprises demineralized water. The other water soluble or water dispersible components of the composition would also be incorporated into the aqueous phase. The oil phase may be any oil conventionally used in cosmetic formulations. Exemplary oils include, but are not limited to fatty alcohols, hydrocarbon oils, natural or synthetic triglycerides, waxes and wax-like materials, esters of long-chain acids and alcohols, silicone oils, fatty acid esters, lanolin and lanolin derivatives, and combinations thereof. Specific examples of oils suitable for use in the invention include those disclosed in U.S. Pat. No. 6,521,217 (Luther et al.), the contents of which are hereby incorporated by reference.

Any emulsifier may be used with the present invention. Suitable emulsifiers include ethoxylated esters of a natural oil derivative such as polyethoxylated esters of hydrogenated castor oil, silicone oil emulsifiers such as silicone polyols, soaps such as, for example, fatty acid soaps and ethoxylated fatty acid soaps, ethoxylated fatty alcohols, optionally ethoxylated sorbitan esters, ethoxylated fatty acids, and ethoxylated glycerides. Specific examples of suitable emulsifiers are disclosed in U.S. Pat. No. 6,290,936 (Ross), the contents of which are hereby incorporated by reference.

Preferred emulsifiers include behenyl alcohol, Steareath-2, PEG-40 stearate, and glyceryl stearate. The emulsions may further comprise any of the thickeners well known in the art. A preferred thickener for use in the emulsions of the present invention is the carbomer available as Carbopol® 940 (Noveon, Ohio).

When formulated as an oil-in-water emulsion, the cosmetically acceptable vehicle preferably comprises 5 to 50% of an oil phase and 30 to 90% of water, each by weight based on the total weight of the emulsion, with the remainder optionally comprising an emulsifier.

The invention imposes no limitation on the amount of color aggregate present in the aqueous phase. Of course, for a particular polymer the upper limit may be imposed by the amount of polymer that is dispersible at low pH. It is within the skill in the art to determine how much polymer is dispersible at a particular pH. Preferably, the color aggregates are present in the aqueous solution between about 1 and about 30% by weight based on the total weight of water. More preferably, the color aggregates are present in the aqueous solution between about 5 and about 15% by weight.

The aqueous attero-chromic compositions prepared according to the methods disclosed herein can be added to a base emulsion immediately after the neutralization step without an intervening purification step. The aqueous attero-chromic compositions are typically added to a base emulsion in an amount ranging from about 0.1 to about 5.0% by dry weight of the color aggregate based on the weight of the base emulsion, and preferably from about 0.5 to about 2.5% by weight.

These emulsions can cover a broad range of consistencies including a thin lotion (which can be suitable for spray or aerosol delivery) to a heavy cream.

In addition to the forgoing, the personal care products of the invention may further comprise sun-screen agents such as, for example, those disclosed in U.S. Pat. No. 5,000,937 (Grollier et al.), U.S. Pat. No. 6,699,461 (Candau), U.S. Pat. No. 6,692,754 (Makimoto et al.), and U.S. Pat. No. 6,290,936 (Ross), the contents of which are hereby incorporated by reference, and/or insect repellants such as, for example, those disclosed in U.S. Pat. No. 6,719,959 (Gonzalez et al.) and U.S. Pat. No. 6,531,118 (Gonzalez et al.), the contents of which are hereby incorporated by reference.

The personal care products of the invention compositions may optionally comprise other active and inactive ingredients, including, but not limited to, excipients, fillers, emulsifying agents, antioxidants, surfactants, film formers, chelating agents, gelling agents, thickeners, emollients, humectants, moisturizers, vitamins, minerals, viscosity and/or rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, colorants, staining agents, depigmenting agents, hypopigmenting agents, preservatives, stabilizers, pharmaceutical agents, photostabilizing agents, and mixtures thereof. In addition to the foregoing, the personal care products of the invention may contain any other compound for the treatment of skin disorders.

The preferred form of the product is a cream that is an oil-in-water emulsion, however, the invention is not so limited. The product form of the present invention may be, for example, an aerosol, cream, emulsion, gel, liquid, lotion, mousse, patch, pomade, powder, solid, spray, stick or towelette. The compositions may also include a vehicle acceptable for topical application to the skin or hair. Examples of such vehicles include, but are not limited to, water and aqueous systems; glycerin; various hydrophilic solvents including alcohols such as ethanol, methanol, propyl and other alcohols; or any combinations thereof. In addition, the vehicle of the compositions according to the present invention can be in the form of a suspension, gel, or solution, formulation. Other suitable topical carriers include an anhydrous liquid solvent such as oil and mono- or polyhydric alcohols; aqueous-based single phase liquid solvent (e.g. hydro-alcoholic solvent system); anhydrous solid and semisolid (such as a gel and a stick); and aqueous based gel and mousse system.

The attero-chromic compositions of the invention are contemplated to be useful in number of commercial products, including but not limited to, oral care products, surfactant/cleaning products, over-the-counter drugs and pharmaceuticals, artificial tanning products, sporting camouflage, foot-care products, liquid and bar soaps, anti-perspirant and deodorant products, fragrance-emitting products, analgesics, insect repellents, poison ivy products including poison ivy blocks, jellyfish protectants, hair care products, shampoos, conditioners, hair colors, hair styling products, hair mascaras, and decorative cosmetics including various make-up products, pressed powder, moisturizers, facial treatment products such as foundations and concealers, mascara, eye liners, body art, lipsticks, lip gloss and lip balms.

### EXAMPLE 1

This example illustrates the preparation of an attero-chromic color aggregate according to the present invention. Table 1 provides a representative formulation for the attero-chromic aggregates.

TABLE 1
ATTERO-CHROMIC AGGREGATES FORMULATION

| Ingredient | Weight % |
|---|---|
| Demineralized water | QS to 100% |
| Carbopol ® ETD 2020¹ | 0.45% |
| Triethanolamine | 0.85% |
| Carbopol ® ETD 1342¹ | 0.40% |
| Iron oxides and methicone² | 3.80% |
| Titanium dioxide | 5.78% |
| Magnesium chloride | 0.20% |

¹Carbopol ® ETD 1342 and Carbopol ® ETD 2020 are acrylates/C10-30 alkyl acrylate crosspolymers available from Noveon, OH;
²Tudor Hydrophobic sold by Kingfisher Colours.

All of the ingredient listed in Table 1 except for triethanolamine were combined in a mixing vessel under vortex mixing. The pH of the aqueous slurry dropped below 5. After the pigments became substantially dispersed in the aqueous slurry, triethanolamine was added as a neutralizing agent to bring the pH to about 7. Mixing was continued until the composition was substantially uniform.

### EXAMPLE 2

This example illustrates the preparation of a base emulsion for use with products comprising the attero-chromic color aggregate according to the present invention. Table 2 provides a representative formulation for an oil-in-water base emulsion.

TABLE 2
BASE EMULSION FORMULATION

| Ingredient | Weight % |
|---|---|
| Demineralized water | QS to 100% |
| Glycerin | 7.50% |
| Carbopol ® 940¹ | 0.50% |
| C12-15 alkyl benzoate | 2.50% |
| Glycerol monostearate | 0.75% |
| Steareth-2 | 0.95% |
| PEG-40 stearate | 0.95% |
| Behenyl alcohol | 0.40% |
| Sodium hydroxide solution 50% | 0.67% |

¹Carbopol ® 940 is a carbomer available from Noveon, OH

The base emulsion components listed in Table 2, excluding sodium hydroxide, were mixed and heated to 75° C. When the mixture became substantially homogenous, the sodium hydroxide solution was added and the emulsion was cooled to 50° C.

### EXAMPLE 3

The aqueous solution comprising the color aggregates of Example 1 was added to the base emulsion of Example 2 without purification or isolation of the color aggregates with mixing. Mixing was continued until the composition was substantially uniform.

The resulting composition had the texture of a cream and was white to off-white in color. When then composition was applied to the skin with rubbing a brownish hue developed which intensified with continued rubbing.

The invention having been described by the foregoing description of the preferred embodiments, it will be understood that the skilled artisan may make modifications and variations of these embodiments without departing from the spirit or scope of the invention as set forth in the following claims.

1. A color-changing composition comprising:
(a) an aggregate comprising:
(1) an anionic polymer; said anionic polymer being at least partially neutralized with a neutralizing agent;
(2) an optionally surface treated non-encapsulated metal oxide colorant non-covalently associated with said anionic polymer; and
(b) a cosmetically or pharmaceutically acceptable vehicle.

2. The color-changing composition of claim 1 wherein said non-encapsulated metal oxide colorant has a positive zeta potential at a pH below about 5 and said anionic polymer has a negative zeta potential at said pH below about 5.

3. The color-changing composition of claim 1 wherein said anionic polymer is water-dispersible at a pH of less than about 5 and water soluble at a pH of between about 5 and about 7.

4. The color-changing composition of claim 1 wherein said anionic polymer is selected from the group consisting of carbomers, polyacrylates, organo-modified acrylates copolymers containing from 3 to 60 carbons, vinyl acrylates, acrylamide polymers and copolymers, and combinations thereof.

5. The color-changing composition of claim 4 wherein said anionic polymer comprises an acrylates/C10-30 alkyl acrylate crosspolymer.

6. The color-changing composition of claim 1 wherein said metal oxide colorant is selected from the group consisting of iron oxide, titanium dioxide, chrome oxide, zinc oxide, and combinations thereof.

7. The color-changing composition of claim 6 wherein said metal oxide colorant is a treated with a surface treatment composition.

8. The color-changing composition of claim 7 wherein said surface treatment composition comprises a material selected from the group consisting of alumina, silica, fumed silica, waxes containing from 3 to 60 carbons, silicones, organosilicones, halosilicones, amino acids and salts and esters thereof, olefins and polymers thereof, fomblins, mineral oil, fatty acids and derivatives thereof, fatty alcohols and derivatives thereof, soaps, metal oxides, inorganic salts, halogenated organic compounds, surfactants, phospholipids, carbonate esters and ethers, and polymers and copolymers thereof.

9. The color-changing composition of claim 8 wherein said surface treatment composition is methicone or a derivative thereof.

10. The color-changing composition of claim 1 wherein said neutralizing agent is selected from the group comprising amines, amino acids, ammonium compounds, hydroxides of alkali and alkaline earth metals, and combinations thereof.

11. The color-changing composition of claim 10 wherein said neutralizing agent is an amine.

12. The color-changing composition of claim 11 wherein said amine is a mono-, di-, or tri-hydroxyalkyl amine.

13. The color-changing composition of claim 1 further comprising an electrolyte.

14. The color-changing composition of claim 13 wherein said electrolyte is a salt of an alkali metal or alkaline earth metal, and combinations thereof.

15. The color-changing composition of claim 14 wherein said electrolyte is selected from the group consisting of NaCl, KCl, CaCl₂, MgCl₂, and combinations thereof.

21. The personal care product of claim 43 wherein said personal care product is a facial treatment product.

22. The personal care product of claim 43 wherein said personal care product comprises an emulsion.

23. The personal care product of claim 22 wherein said emulsion is selected from the group consisting of oil-in-water, water-in-oil-in-water, oil-in-oil, and polyol-in-oil emulsions.

24. The personal care product of claim 23 wherein said emulsion is an oil-in-water emulsion.

25. A method of making a color-changing composition comprising the steps of:
(a) providing and aqueous slurry comprising an anionic polymer and an optionally surface treated metal oxide colorant, said aqueous slurry having a pH of less than about 5 and said anionic polymer comprising between about 0.1 to about 5% by weight of said aqueous slurry;
(b) adding a neutralizing agent to said aqueous slurry in an amount sufficient to raise the pH of said aqueous slurry to between about 5 and 7.

26. The method of claim 25 wherein said anionic polymer is selected from the group consisting of carbomers, polyacrylates, organo-modified acrylates copolymers containing from 3 to 60 carbons, vinyl acrylates, acrylamide polymers and copolymers, and combinations thereof.

27. The method of claim 26 wherein said anionic polymer comprises an acrylates/C10-30 alkyl acrylate crosspolymer.

28. The method of claim 25 wherein said metal oxide is selected from the group consisting of iron oxide, titanium dioxide, chrome oxide, zinc oxide, and combinations thereof.

29. The method of claim 25 wherein said inorganic colorant is a treated with a surface treatment composition.

30. The method of claim 29 wherein said surface treatment composition comprises a material selected from the group consisting of alumina, silica, fumed silica, waxes containing from 3 to 60 carbons, silicones, organosilicones, halosilicones, amino acids and salts and esters thereof, olefins and polymers thereof, fomblins, mineral oil, fatty acids and derivatives thereof, fatty alcohols and derivatives thereof, soaps, metal oxides, inorganic salts, halogenated organic compounds, surfactants, phospholipids, carbonate esters and ethers, and polymers and copolymers thereof.

31. The method of claim 30 wherein said surface treatment composition is methicone or a derivative thereof.

32. The method of claim 25 wherein said neutralizing agent is selected from the group comprising amines, amino acids, ammonium compounds, hydroxides of alkali and alkaline earth metals, and combinations thereof.

33. The method of claim 32 wherein said neutralizing agent is an amine.

34. The method of claim 33 wherein said amine is a mono-, di-, or tri-hydroxyalkyl amine.

35. The method of claim 25 wherein step (a) further comprises the addition of an electrolyte to said aqueous slurry.

36. The method of claim 35 wherein said electrolyte comprises from about 0.1 to about 3% by weight of said aqueous slurry.

37. The method of claim 35 wherein said electrolyte is a salt of an alkali metal or alkaline earth metal, and combinations thereof.

38. The method of claim 37 wherein said electrolyte is selected from NaCl, KCl, CaCl₂, MgCl₂ and combinations thereof.

39. A method of making a color-changing composition comprising the steps of
(a) providing and aqueous slurry having a pH of less than about 5, said aqueous slurry comprising:
(i) an anionic acrylates/C10-30 alkyl acrylate crosspolymer; wherein said anionic polymer is water-dispersible at a pH of less than about 5 and water soluble at a pH of between about 5 and about 7, said anionic polymer comprising between about 0.1 to about 5% by weight of said aqueous slurry;
(ii) an optionally surface treated metal oxide colorant selected from the group consisting of titanium dioxide, iron oxides, and combinations thereof; and
(iii) an electrolyte; said electrolyte comprising between about 0.1 to about 3% by weight of said aqueous slurry; and
(b) adding a neutralizing agent to said aqueous slurry in an amount sufficient to raise the pH of said aqueous slurry to between about 5 and 7; wherein said neutralizing agent comprises an amine.

40. The color-changing composition of claim 1 wherein the ratio of said optionally surface treated non-encapsulated metal oxide colorant to said anionic polymer is about 30:1 to about 1:1.

41. The color-changing composition of claim 40 wherein the ratio of said optionally surface treated non-encapsulated metal oxide colorant to said anionic polymer is about 15:1 to about 5:1.

42. A color-changing composition comprising:
(a) a color aggregate comprising:
(a) an anionic polymer that is at least partially neutralized with an amine neutralizing agent, said anionic polymer comprising an acrylates/C10-30 alkyl acrylate crosspolymer; said anionic polymer being water-dispersible at a pH of less than about 5 and water soluble at a pH of between about 5 and about 7; and
(b) a non-encapsulated optionally surface treated colorant selected from the group consisting of titanium dioxide, iron oxides, and combinations thereof; and
(b) a cosmetically or pharmaceutically acceptable vehicle.

43. A personal care product comprising the color changing composition of claim 42.

44. The personal care product of claim 43 wherein said personal care product is selected from the group consisting of sun-care products, oral care products, surfactant products, cleaning products, over-the-counter drugs and pharmaceuticals, artificial tanning products, sporting camouflage, foot-care products, liquid and bar soaps, anti-perspirant and deodorant products, fragrance-emitting products, analgesics, insect repellents, poison ivy products, jellyfish protectants, hair care products, shampoos, facial treatment, conditioners, hair colors, hair styling products, hair mascaras, make-up products, pressed powder, mascara, eye liners, lipsticks, lip gloss and lip balms.
